(19) United States
(12) Patent Application Publication
(10) Pub. No.: US 2003/0017578 A1
(43) Pub. Date: Jan. 23, 2003

(54) Apparatus for transferring molecules into cells

(75) Inventor: Friedrich Ueberle, Gilching (DE)

Correspondence Address: KING & SPALDING, 191 PEACHTREE STREET, N.E., ATLANTA, GA, 30303-1763, US

(73) Assignee: Dornier Medizintechnik GmbH, Wessling (DE)

(21) Appl. No.: 10/177,823

(22) Filed: Jun. 21, 2002

Related U.S. Application Data
(63) Continuation of application No. PCT/DE00/04631, filed on Dec. 23, 2000.

(30) Foreign Application Priority Data
Dec. 23, 1999 (DE) 19962904.8-41
Dec. 20, 2000 (DE) 10063942.9

(51) Int. Cl. C12M 3/00

(52) U.S. Cl. 435/285.1

(57) ABSTRACT

A method for the transfer of molecules into cells and apparatus for performing the method.

A medium in which cells and molecules to be transferred into the cells are located is moved within a hollow cylindrical device for generating acoustic, cavitation-inducing pulses relative to an area which the acoustic pulses occur in a focused manner.

The present invention relates to an apparatus according to the preamble of claim 1.

German application 19834612.3-41 discloses an apparatus and a method for the intracellular transfer of oligonucleotides under the action of shock waves. In the invention described therein, a sample container is put with the molecules to be transferred and the target cells into some kind of water bath and is exposed to a predetermined number of shock waves. The molecules and the target cells are positioned in a medium which guides sound waves and in which the vital functions of the target cells are maintained. Said shock waves produce a so-called cavitation which makes the target cells transiently permeable to the molecules to be transferred.

Subsequently, the sample container is exchanged. This has the drawback that the molecules to be transferred and the target cells must be put into the sample container and said container must then be placed into the water bath, which is relatively complicated. This results in a quantitative limitation of the target cells doped with molecules, which makes the known prior art unsuitable for commercial use, e.g. in the pharmaceutical industry.

It is known from DE 3821354 to generate cavitation in larger volumes. A so-called sonotrode is there used as a generator for the cavitation. However, it is also known from said publication that even high-performance sonotrodes as such cannot supply large volumes in an efficient way with a short-term cavitation; therefore, additional activators are there introduced into the suspension and made resonant, in turn, and then produce cavitation in their surroundings in the resonant state. A drawback is here that said activators must again be removed from the suspension.

Furthermore, it is known from WO 99/58637 to produce cavitation in a volume by means of arrays of piezoelectric transducers. Controllable, constructive and destructive sound interferences are produced by piezoelectric transducers driven in a phase-shifted way, with the aim to achieve a kind of focusing at places of interference maxima. However, this has the drawback that the focusing area produced in this way only comprises small volumes and that the technical efforts for driving and controlling the piezoelectric transducer arrays are very high.

To effect a transfection on the one hand and to destroy only a minimum amount of cells on the other hand, it has surprisingly been found to be of particular advantage when the acoustic pulses are only operative for a short period of time, but do not exceed the cavitation threshold of the liquid during operation.

In known devices for producing such cavitation-generating acoustic pulses operating with so-called sonotrodes, the serious drawback is that the sonotrodes must first be made resonant to produce vibrations which have such a high amplitude that the cavitation threshold is exceeded. Individual pulses are thus not possible, which is disadvantageous. This leads to the additional drawback that an excessive “dose” of cavitation events acts on the cells, thereby destroying the cells.

The conventional design of the sonication device with sonotrodes additionally promotes the formation of foam and aerosol, whereby the reproducibility of the results of sonification is reduced.

In the apparatus which is known from WO 99/58637 and includes arrays of piezoelectric transducers driven in phase-shifted fashion, the focus will change as soon as cavitation occurs, whereby the focusing area can only be predicted stochastically.

It is therefore the object of the invention to provide an apparatus, whereby molecules can be transferred into target cells and the drawbacks of the prior art are overcome and an efficient, quantitatively strongly enhanced production is made possible.

It is the object of the invention to provide an apparatus by which cavitation can be produced, said cavitation being, on the one hand, sufficient to make cells transiently permeable to molecules and having, on the other hand, only such a short duration that the cells are substantially not damaged in a lasting way and that in particular an efficient, quantitatively strongly enhanced production is made possible.

Said object is achieved by the characterizing features of the respective main claims.

The respective subclaims concern developments and/or particularly advantageous developments of the invention.

The ideas which led to the creation of this invention adopted the finding that a hollow cylindrical device into which a plurality of cophasal acoustic pulses are introduced radially from the outside produces a reproducible zone of transient cavitation events in the area around the rotational axis of the hollow cylinder. The ideas leading to the creation of this invention started on the assumption that it is irrelevant to the transfer of the molecules into the target cells how great the whole amount of the medium is within which the molecules to be transferred and the target cells are located provided that parts from the total amount successively pass into the operative area of the acoustic pulses and the target cells contained therein are thus subjected to a treatment.

To treat a volume of said medium which is large in relation to the operative area of the focused acoustic pulses, a relative movement takes place according to the invention between the source of the focused acoustic pulse and the medium to be treated, whereby continuously new areas of the medium that have not been treated with focused acoustic pulses before are treated.

Said relative movement can now take place by moving the source relative to a fixed liquid container or by moving the liquid container relative to a stationary source.

Accordingly the medium with the target cells and the molecules to be transferred can also move through a line system while it is under the influence of the focused acoustic pulses.

In general, there must prevail a relative speed between the medium and the source of acoustic pulses that is adapted to the spatial extension of the operative area of the acoustic pulses and the repetition frequency of the acoustic pulses. The parameters must be adapted to one another such that the target cells during their movement through the line system are exposed at least to the predetermined number of pulses.

The invention shall now be explained in more detail with reference to a possible embodiment.

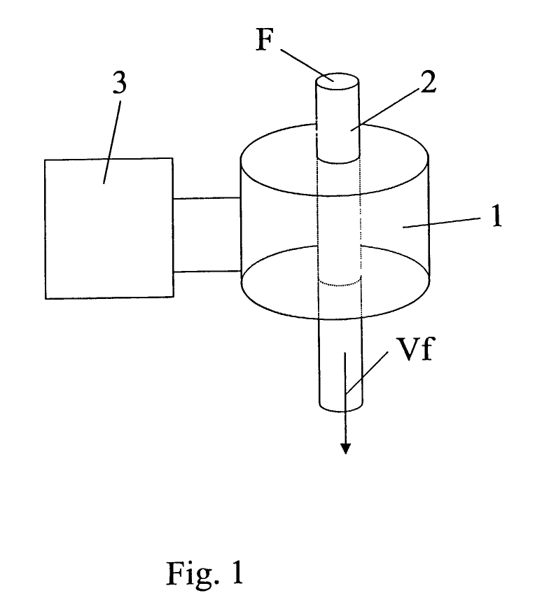

FIG. 1 shows a possible embodiment of the invention. Acoustic pulses are generated by means of a substantially hollow-cylindrical source 1 through which a tubular line 2 is passed. Said line 2 contains the medium. In the illustrated case this is a liquid F having a viscosity permitting a flow through line 2.

The source 1 is configured such that it focuses the acoustic pulses in its interior and thus in the area in which line 2 extends. The focus extends substantially in axial direction. The area of the focus is the so-called operative area of the acoustic pulses and will therefore be called focusing area in the following. In the illustrated embodiment source 1 is shaped as a rotationally symmetrical cylinder. The wall of line 2 is permeable to acoustic pulses—at least in the area of the focus. A suitable coupling medium which transmits the acoustic pulses from source 1 to line 2 is positioned between source 1 and line 2.

The use of a plurality of piezoelectric elements which are each oriented in concentric fashion in the direction of the rotational axis and are excited at the same time in phase is intended as excitation principle for the acoustic pulses. It is thereby possible to produce an elongated focusing area.

Instead of a plurality of piezoelectric elements oriented in concentric fashion relative to the rotational axis, it is also possible to realize the cylindrical source by a plurality of adjacent piezoelectric rings whose point of center is positioned on the rotational axis.

Likewise, it is also possible to use magnetostrictive elements as actors for generating the acoustic pulses.

According to a further embodiment of the invention the source 1 is designed as a hollow body of a semicircular cross-section as a rather trough-like body. In these embodiments a focusing area is obtained which extends approximately over the length of the source. The line in which liquid F is located extends through said elongated focusing area. This has the advantage that the source can be coupled to an existing line and that the line need not first be laid through the hollow cylindrical source 1.

It is also possible to provide a gap in the hollow cylindrical source, the line being introducible through said gap into the interior of the hollow cylindrical body. In this embodiment the source can also be coupled in an advantageous manner to an existing line.

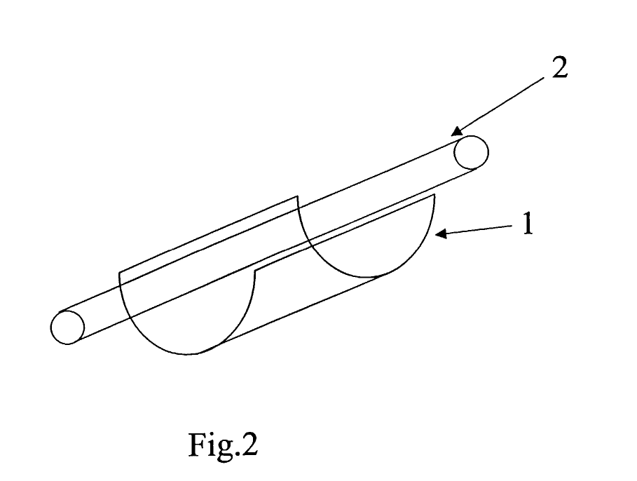

The configuration of a source 1 according to FIG. 2 with a semicircular cross-section offers the advantage that the source can easily be attached from the side to a line, without the need for laying the line through the interior of the source.

It is also possible to design the hollow body such that it can be divided and then be placed around the line from both sides.

It is also possible to use an exploding wire as the source of acoustic pulses, said wire being stretched along the focusing line of an elliptically or parabolically shaped hollow tube.

Likewise, it is possible to use a coil for excitation, the coil acting on a membrane positioned inside the coil, thereby generating an inwardly directed acoustic pulse.

Acoustic pulses are radiated by the source 1 onto the liquid F, thereby producing at said place short-term conditions (pressure or vacuum of an adequate intensity, cavitation) for effecting a transfer of the molecules into the target cells.

It has been found that a transfer of the molecules into the cells is possible with wide pressure or vacuum ranges. The pressure ranges are here between 10 MPa and 150 MPa. The vacuum ranges are at −5 MPa to 50 MPa. The intensity is between 0.5 mJ/mm² and 5.0 mJ/mm².

Moreover, it has been found that a longer duration of the cavitation events does not help to increase the transfection rate but rather that the cells are more and more damaged in a disadvantageous way with an increasing duration of the cavitation events.

Therefore, only a short-term cavitation event is produced with the source according to the invention.

The flow rate Vf of liquid F can be varied by a pump, which is not drawn for the sake of clarity.

Source 1 is excited in pulsed fashion by a unit 3. The target cells and the molecules to be transferred are exposed to a predetermined number of acoustic pulses of a predetermined intensity during flow through line 2.

Due to the swirls caused by flow and cavitation inside line 2, all cells of the suspension pass, stochastically speaking, into the operative area of the short-term cavitation sufficiently often while flowing through the source and are thereby made transiently permeable.

Likewise, it is possible to make the liquid F flow in pulsating or also cycled fashion through line 2. The liquid F is here always conveyed such that the area which was just in front of the source 1 in the line is conveyed further to such an extent that said area then comes to stop approximately at the end of source 1. While the liquid area is positioned inside the source 1 in the focus of the acoustic pulses, a predetermined number of acoustic pulses are output by which the transfer of molecules into the target cells is excited.

The acoustic pulses which act on the liquid or, in general terms, on the medium may consist of ultrasonic pulses or of one or several successive shock waves.

It has been found that the transfer of the molecules into the cells depends on the number of ultrasonic pulses or shock waves. The transfection rate rises with a higher number of ultrasonic pulses or shock waves. Likewise, the number of the cells damaged by the treatment increases with the number of ultrasonic pulses or shock waves.

The intensity (mass unit mJ/mm²) of the ultrasonic pulses or shock waves also has an influence on both the number of the intracellularly transferred molecules and on the damage of the cells. An increasing intensity results in an increase for both the number of the intracellularly transferred molecules and the damaged cell.

By analogy with the described movement of the medium through the line, it is also possible to realize the relative movement according to the invention between the source of acoustic pulses and the medium in which the molecules and the target cells are positioned by moving a container in which the medium is located. The source of the acoustic pulses is here stationary and the container is moved.

The container may have any desired shape, but it is decisive that the movement of the container takes place such that the whole volume of the container successively passes into the operative area of the acoustic pulses.

It is also possible to fill and seal the container at a sterile place and then to treat said container in a “non-sterile area” with the acoustic pulses.

Likewise, it is possible to make the container stationary and to move one or also several sources of acoustic pulses such that the whole volume of the container successively passes into the operative area of the acoustic pulses.

All movements of the medium relative to the source of acoustic pulses can take place continuously or also in cycled fashion.

According to a development of the invention, the molecules to be transferred are first directly supplied in front of the focusing area to the medium in which the target cells are positioned.

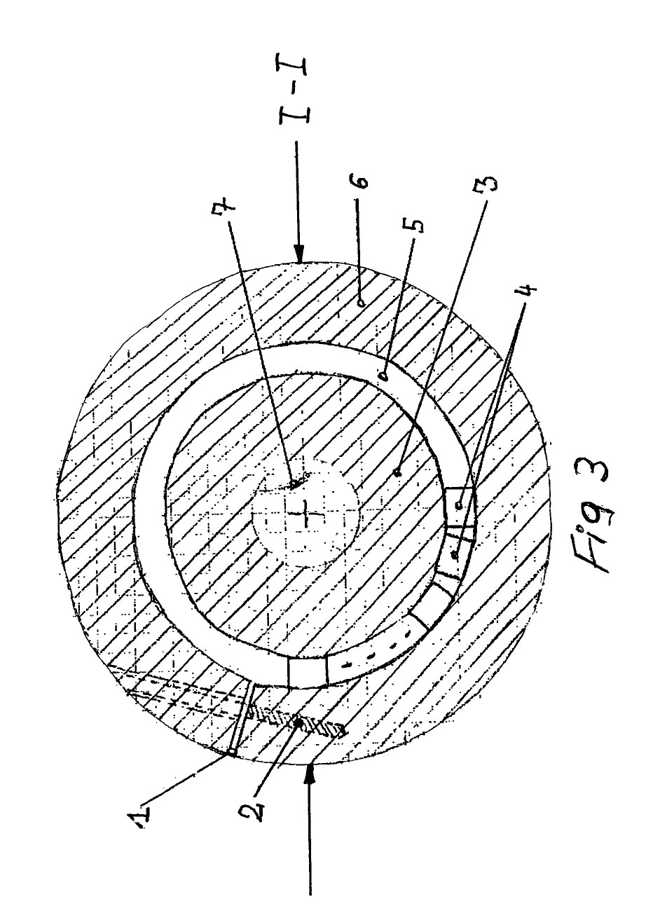

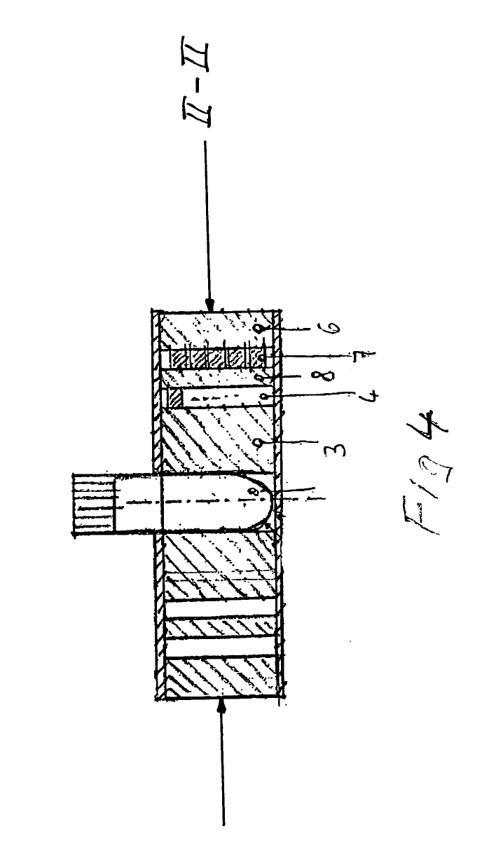

A particularly advantageous construction of the source is shown in FIGS. 3 and 4.

The source shown in FIG. 3 consists of an inner ring 3 which can receive the line (here not shown) or small sample tube in its free inner space 7.

Piezoelectric elements 4 are arranged in distributed fashion around the outer circumference of the inner ring 3. Only a few of said piezoelectric elements 4 are shown in this illustration for reasons of clarity.

The piezoelectric elements 4 are held by a further ring 6 at their side facing away from the inner ring 3. The outer ring 6 is designed as a clamping ring. Its inner diameter can be changed via a clamping screw 2. Gap 1 is here also changed.

The piezoelectric elements 4 are thereby firmly clamped between the inner ring 3 and the outer clamping ring 6. A good acoustic transmission between the piezoelectric elements 4 and rings 3 and 6 is thereby ensured.

FIG. 4 shows a further version of the apparatus according to the invention. The piezoelectric elements 4 are there arranged in the way known from FIG. 3 around the inner ring 3. An intermediate ring 8 is arranged at its side facing away from the inner ring 3. Piezoelectric elements 7 are again arranged on said intermediate ring 8. Said piezoelectric elements 7 are followed by the clamping ring 6, which is already known from FIG. 3.

1. An apparatus for transferring molecules into cells, wherein a medium (F) in which the molecules to be transferred and the target cells are contained can be exposed in the focusing area of a source of acoustic pulses to said acoustic pulses, characterized in that
said source comprises an at least approximately line-shaped focusing area in which said acoustic pulses exceed a predetermined pressure or negative pressure and/or a predetermined intensity, and
a device is provided by which a relative movement between said medium (F) and said focusing area is performed.

2. The apparatus according to claim 1, characterized in that
a line (2) is provided by which said medium (F) can be transported and said line is passed through the focusing area of said source (1).

3. The apparatus according to claim 1 or 2, characterized in that
said source (1) is designed as a substantially hollow-cylindrical body and said focusing area extends along the center axis of said hollow-cylindrical body.

4. The apparatus according to claim 1 or 2, characterized in that
said source (1) of acoustic pulses is designed as a body having a semicircular cross-section and said focusing area extends along the center line of said semicircle.

5. The apparatus according to any one of claims 1 to 4, characterized in that
said source (1) comprises a plurality of piezoelectric elements which can be excited simultaneously and in phase.

6. The apparatus according to any one of claims 1 to 5, characterized in that
said source (1) consists of a plurality of adjacent piezoelectric rings.

7. The apparatus according to any one of claims 1 to 6, characterized in that
there is provided an access to said line (2) through which the molecules to be transferred can be supplied to said medium (F) in which said target cells are positioned.

8. The apparatus according to any one of claims 1 to 7, characterized in that
a plurality of acoustic transducers are mounted on a common carrier and a mechanical element (6) presses said acoustic transducers (4) onto said carrier (3).

9. The apparatus according to claim 8 characterized in that
said mechanical element (6) contacts said acoustic transducers (4) at the side facing away from said carrier (3).

9. The apparatus according to claim 8, characterized in that
said mechanical element (6) is designed in the form of a clamping ring.

10. The apparatus according to claim 8, characterized in that
said carrier (3) and said mechanical element (6) are of a ring-like construction.

11. The apparatus according to any one of claims 8 to 10, characterized in that
said acoustic transducers are arranged in a plurality of layers having a different distance from said carrier (3).

12. The apparatus according to claim 11, characterized in that
said different layers of acoustic transducers are separated by separation layers.
